(19) United States
(12) Patent Application Publication
(10) Pub. No.: US 2026/0031184 A1
(43) Pub. Date: Jan. 29, 2026

(54) A METHOD TO OPTIMISE TRANSCRIPTOMIC SIGNATURES

(71) Applicant: IMPERIAL COLLEGE INNOVATIONS LIMITED, London (GB)

(72) Inventors: Jesus RODRIGUEZ MANZANO, London (GB); Dominic COOTE, London (GB); Heather JACKSON, London (GB); Luca MIGLIETTA, London (GB); Myrsini KAFOROU, London (GB)

(21) Appl. No.: 19/099,027

(22) PCT Filed: Jul. 20, 2023
(86) PCT No.: PCT/GB2023/051904

§ 371 Date: Jan. 27, 2025

(30) Foreign Application Priority Data
Jul. 28, 2022 (GR) 20220100614
Aug. 10, 2022 (GB) 2211707.1

(51) Int. Cl. G16B 25/10; G16H 50/20

(52) U.S. Cl. CPC G16B 25/10; G16H 50/20

(57) ABSTRACT

The present application relates to a method of obtaining an optimised transcriptomic signature for identification of a biological condition. The optimised transcriptomic signature being optimised for detection via a particular test platform of a plurality of test platforms, each test platform being associated with at least one requirement. The method comprises receiving transcriptomic data obtained from a first set of subjects, wherein a first subset of subjects in the first set of subjects do have the biological condition, and a second subset of subjects in the first set of subjects do not have the biological condition processing the transcriptomic data to identify a plurality of candidate features, each candidate feature being suitable for use in identifying the biological condition. The method further comprises processing, based on at least one first requirement associated with a first test platform of the plurality of test platforms, the plurality of candidate features. The method further comprises outputting, based on the processing of the plurality of candidate features, the optimised transcriptomic signature for identification of a biological condition being optimised for detection via the first test platform, the optimised transcriptomic signature comprising at least one feature of the plurality of candidate features. The application also relates to a system comprising a memory for storing computer-readable instructions; and one or more processors for executing the computer readable instructions to perform the method.

### TECHNICAL FIELD

This disclosure relates to a method and system of obtaining a transcriptomic signature that is optimised for a particular test platform, and enables optimal discrimination in the presence of a host-response indicative of a biological condition, such as a pathogen or a biological stage.

### BACKGROUND

A new paradigm of diagnostic testing is needed to guide clinical care of patients where clinical presentation of, for example, pathogen detection is insufficient to guide treatment, prognosis and screening. High-throughput host transcriptomics, such as RNA sequencing (RNAseq) or Microarrays, offers an alternative to traditional pathogen-based diagnostic processes. In some embodiments, diagnostic assays based on transcriptomic signatures may be more desirable than pathogen based as they can be a more generalised solution (e.g., unique sample source for multiple pathogens and/or biological conditions). Rather than having several confirmatory tests for several different pathogens, each of which requires different samples and technologies, a single transcriptomic signature may be used.

Omics data includes data from high-throughput biochemical assays that measure molecules of the same type from a biological sample. For example, this may include genomics profile DNA, transcriptomics, proteomics and metabolomics. Transcriptomics in particular is the study of the complete set of RNA transcripts that are produced by the genome, under specific circumstances or in a specific cell, obtained using these high-throughput methods. The specific circumstances may be the presence of a pathogen that induces a host response.

However, omics data-based methods can be challenging to be directly used for clinical diagnostics due to a lengthy laboratory and analytical stages, which can be both expensive and time-consuming. In particular, translation of high-throughput sequencing data, such as RNAseq, to a PCR-based platform is not trivial.

PCE-based diagnostic tests are a much more viable alternative to host transcriptomics as they can feasibly be integrated into existing clinical practises. This reduces the time and expense needed for diagnosis of a particular pathogen, as well as reducing the laboratory resources required.

However, there is a gap in moving from the highly accurate host-response data from high-throughput methods (such as RNAseq analysis) to a sensitive, specific and targeted host response tests, such as RT-qPCR or RT-qLAMP. One reason for this gap is the compromised performance of the diagnostic signatures (such as transcriptomnic signatures) when they are transferred to simpler detection and quantification platforms. The likelihood of successful cross-platform validation of features is therefore low.

The present invention seeks to address these and other disadvantages encountered in the prior art by providing an improved and/or tailored signature for detection of a biological condition via a particular test platform of a plurality of test platforms.

### SUMMARY

An invention is set out in the independent claims. Optional features are set out in the dependent claims.

According to an aspect, there is provided a computer-implemented method of obtaining an optimised transcriptomic signature for identification of a biological condition. The optimised transcriptomic signature is optimised for detection via a particular test platform of a plurality of test platforms, each test platform being associated with at least one requirement associated with the respective test platform. The method comprises receiving transcriptomic data obtained from a first set of subjects. A first subset of subjects in the first set of subjects do have the biological condition, and a second subset of subjects in the first set of subjects do not have the biological condition. The method further comprises processing the first set of transcriptomic data to identify a plurality of candidate features of the transcriptomic data, each candidate feature being suitable for use in identifying the biological condition. The method further comprises processing, based on at least one first requirement associated with a first test platform of the plurality of test platforms, the plurality of candidate features. The method further comprises outputting, based on the processing of the plurality of candidate features, the optimised transcriptomic signature optimised for detection via the first test platform. The optimised transcriptomic signature comprises at least one feature of the plurality of candidate features.

Optionally, processing the plurality of candidate features of the transcriptomic data comprises filtering the candidate features based on the at least one first requirement.

Optionally, the filtering further comprises determining that a first feature of the plurality of candidate features is unsuitable, based on the first requirement, and based on the determining, removing the first feature from the plurality of candidate features.

Optionally, the filtering further comprises determining that a first feature of the plurality of candidate features is essential, based on the first requirement, and based on the determining, including the first feature in the plurality of candidate features.

Optionally, at least one of the plurality of requirements is based on instrumentation of the test platform. Optionally, the at least one requirement based on instrumentation relates to at least one of: resolution, depth coverage, sensitivity, reaction volume, number of fluorescent channels, real-time quantification, end-point quantification, dynamic range of quantification, ability to perform temperature gradient, required preamplification step, sample preparation, and bias detection of a given target. Optionally, at least one of the plurality of requirements is based on chemistry of the test platform. Optionally, the at least one requirement based on chemistry relates to at least one of: primer and sequence target GC content, primer and sequence target length, sequence target melting temperature, maximum and minimum primer melting temperature, maximum and minimum primer 3′ clamp, maximum and minimum primer hairpin melting temperature, maximum and minimum primer cross-dimer melting temperature, maximum and minimum distances between primers.

Optionally, a sample type is associated with the transcriptomic data obtained from the first set of subjects, and at least one of the plurality of requirements is based on the sample type.

Optionally, the first test platform comprises an amplification method. Optionally, the plurality of test platforms includes at least two of: PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR) allele-specific PCR (AS-PCR), intersequence-specific PCR (ISS-PCR), whole genome amplification (WGA), inverse PCR, or thermal asymmetric interlaced PCR (TAIL-PCR), Strand Displacement Amplification (SDA), Transcription Mediated Amplification (TMA), Nucleic Acid Sequence Based Amplification (NASBA), Recombinase Polymerase Amplification (RPA), Rolling Circle Amplification (RCA), Ramification Amplification (RAM), Helicase-Dependent Isothermal DNA Amplification (HDA), Circular Helicase-Dependent Amplification (CHDA), Loop-Mediated Isothermal Amplification (LAMP), Single Primer Isothermal Amplification (SPIA), Signal Mediated Amplification of RNA Technology (SMART), Self-Sustained Sequence Replication (3SR), Genome Exponential Amplification Reaction (GEAR) and Isothermal Multiple Displacement Amplification (IMDA).

Optionally, the method further comprises filtering the plurality of candidate features based on statistical significance measures.

Optionally, the method further comprises applying a feature selection algorithm.

Optionally, the transcriptomic data comprises one of: RNA-sequencing data, gene counts, exon counts, or microarray data.

Optionally, the first test platform is an amplification method, and wherein the method further comprises identifying optimal regions of the transcriptomic data, wherein the optimal regions are regions that should be targeted by at least one primer or probe to enable optimal discrimination when the biological condition is detected via the first test platform.

Optionally, the method further comprises outputting an optimised primer design.

Optionally, the optimised transcriptomic signature is optimised for at least one of efficiency, specificity, and accuracy when the biological condition is detected via the first test platform.

Optionally, identification of the biological condition comprises diagnosing a disease.

Optionally, identification of the biological condition comprises determining a prognosis of a disease.

Optionally, identification of the biological condition comprises screening for a disease.

According to an aspect, there is provided a computer-readable medium comprising computer-readable instructions for performing any of the steps disclosed herein.

According to an aspect, there is provided a system comprising a memory (114, 116) for storing instructions, and further comprising one or more processors for executing the instructions stored in the memory for performing any of the steps disclosed herein.

### FIGURES

Specific embodiments are now described, by way of example only, with reference to the drawings, in which:

FIG. 1 depicts a computer-implemented method of obtaining an optimised transcriptomic signature for identification of a biological condition.

FIG. 2 depicts a workflow of exon selection based on RNAseq data analysis.

FIG. 3 depicts assay design strategy and performance in qPCR instrument.

FIG. 4 depicts exon abundance for the gene biomarker of interest in the patient groups with definite bacterial infection, definite viral infection, and a healthy control group.

FIG. 5 depicts a schematic showing how validation platform constraints were incorporated into the signature discovery process.

FIG. 6 depicts the selection of four genes using log-fold change.

FIG. 7 depicts the effect of assay constrains on the gene/exon selection.

FIG. 8 depicts a block diagram of one implementation of a computing device within which a set of instructions, for causing the computing device to perform any one or more of the methodologies discussed herein, may be executed.

### DETAILED DESCRIPTION

At a high level, the present application relates to a computer-implemented method of obtaining an optimised transcriptomic signature for identification of a biological condition, stage, of event (such as the presence of a pathogen, or a symptom, for example inflammation). The optimised transcriptomic signature is optimised for detection via a particular test platform of a plurality of test platforms, and the method involves taking into account various requirements associated with the test platforms, such as specific temperature requirements, amplicon length requirements, etc.

A number of test platforms may be used for identification of a biological condition, or to give information related to the biological condition, for example by determining a prognosis. The optimised transcriptomic signature may be for at least one of: screening for a disease, for diagnosing a disease, or for determining a prognosis of a disease. In conventional methods, sequencing data such as RNAseq or microarray data is analysed to produce a diagnostic signature indicative of a particular biological condition. This diagnostic signature can be used as the basis of a variety of diagnostic tests performed on one of many different possible testing platforms, such as PCR and LAMP. However, the present inventors have realised that a diagnostic signature based on sequencing data alone may be unsuitable for some diagnostic test platforms, or may not be the optimal signature for that platform. Depending on which test platform is selected, a particular signature may not be ideal for that platform. This may be due to the chemistry or the instrumentation of the test platform.

It is evident that there is a need for a method that optimises the diagnostic signature for the test platform that will be used in order to detect the biological condition of interest. Outputting an optimised transcriptomic signature based on the requirements of a test platform, as done in methods of the present disclosure, means that the cross-platform translation is improved, and the signature is optimised for detection via that particular test platform. There is therefore improved detection of the biological condition when using that signature for that particular test platform.

### Method of Outputting an Optimised Transcriptomic Signature

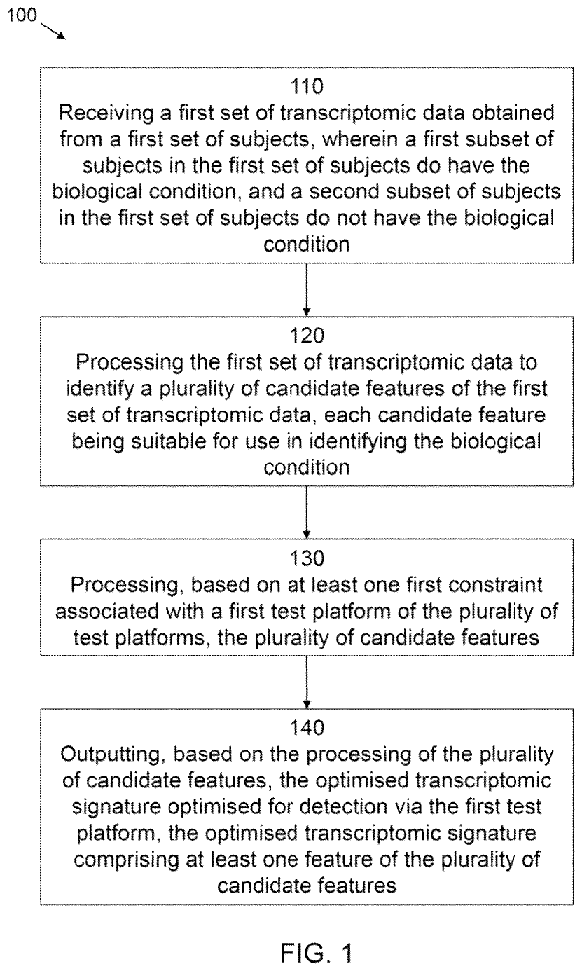

FIG. 1 depicts a computer-implemented method 100 of obtaining an optimised transcriptomic signature for identification of a biological condition. The RNAseq data (or any other high-throughput omics' technologies, such as microarray) may be used to select optimal features (such as genes and/or exons) in order to output an optimised signature.

At step 110, a first set of transcriptomic data obtained from a first set of subjects is received. A first subset of subjects in the first set of subjects do have the biological condition, and a second subset of subjects in the first set of subjects do not have the biological condition. For example, if the biological condition relates to a pathogen, then the first subset of subjects have been exposed to the pathogen, and the second subset of subjects have not been exposed to the pathogen. In a further example, if the biological condition relates to a biological state such as inflammation, then the first subset of subjects may have be experiencing inflammation, and the second subset of subjects may not be.

In some implementations, the data received is high-throughput omic data. Omic data could be measurements of RNA transcripts (transcriptomics), proteins (proteomics), metabolites (metabolomics). Preferably, the ‘omic data is transcriptomic data. The data may be from patients with the diseases of interest, in addition to a group of comparator patients that the disease group of interest needs to be distinguished from.

The data received at step 110 may include, but is not limited to, RNA-sequencing (RNAseq) gene counts, exon counts, or microarray data. The disease groups of interest may be, for example, bacterial and/or viral infections.

At step 120, the first set of transcriptomic data is processed to identify a plurality of candidate features of the first set of transcriptomic data, each candidate feature being suitable for use in identifying the biological condition. A candidate feature may be a single gene, a single exon, a plurality of genes, or a plurality of exons.

Processing to identify a plurality of candidate features may comprise identifying a plurality of features that are significantly differently expressed for the biological condition. For example, the candidate features may be genes significantly differentially expressed between the transcriptomic data for the first subset of subjects and the transcriptomic data for the second subset of subjects. Analysis of changes in read count or expression levels between two conditions (for example, a biological condition and a lack of a biological condition) may be used to identify candidate features.

Differential expression or abundance analysis may be used to identify features that are significantly different between the disease group of interest and comparator group or groups. Typically, this reduces the number of features by 10-fold. This step may be carried out using one or more of: DESeq2, Limma, and EdgeR. In an example, this analysis filters the number of features from approximately 25,000 genes to approximately 200-5,000 features using differential expression analysis. This step advantageously reduces the search space, excluding potentially noisy features and retaining features with diagnostic potential. The identification of an optimised diagnostic signature is therefore more efficient, and uses fewer computational resources.

Optionally, identifying a plurality of candidate features may comprise filtering features by statistical methods such as p-values and log 2 fold-change values. Thresholds may be introduced, for example based on Benjamini-Hochberg (BH) adjusted p-value, log fold-change (LFC) and base mean counts.

Optionally, identifying a plurality of candidate features may comprise applying a feature selection algorithm. Feature selection methods may be used to identify a small number of features (for example, up to 10 features) that when combined, can distinguish between the disease group of interest and the comparator group(s). This may be done using one or more of: feature selection partial least squares (FS-PLS), Lasso and Elastic Net regression, and random forest.

In some implementations, the plurality of candidate features may be selected from a combination of filtering methods, such as at least one first candidate feature that is significantly differently expressed for the biological condition and at least one second candidate feature that is selected using a feature selection algorithm.

At step 130, the plurality of candidate features are processed based on at least one first constraint associated with a first test platform of the plurality of test platforms. For example, thresholds related to GC content or base mean counts may be applied in order to optimise likelihood of cross-platform translation. In particular, considering primer design constraints as a result of the chemistry of the molecular platform in addition to highly discretionary features advantageously allows for improved cross-platform translation.

In an example where a first candidate feature is significantly differently expressed between the first and the second set of subjects, and a second candidate feature is selected using a feature selection algorithm, at least one of these candidate features may not be suitable based on the at least one first constraint. If the test platform is LAMP, a constraint may be that the total amplicon length is at least 200 base pairs for optimised detection of a biological condition. Therefore, if the exon length of a particular region of interest does not satisfy that constraint, it may not be suitable for the translation of the RNA signature to LAMP, or may not be optimised for translation to LAMP. In an embodiment, that exon may be excluded from the plurality of candidate features, such that an optimised transcriptomic signature does not include that exon. In a further example, a constraint may be related to the melting temperature (Tₘ) of a primer. For each candidate feature in the plurality of candidate features, multiple assays may be designed, and be evaluated. For example, at least one of sequence target melting temperature, maximum and minimum primer melting temperature, and maximum and minimum primer cross-dimer melting temperature may be considered to determine optimise the candidate features in the transcriptomic signature for a particular melting temperature or temperature range.

Optionally, the processing may comprise filtering the plurality of candidate features, such that features that are not optimised for detecting the biological condition on the test platform are not included in the optimised signature.

Optionally, to the method further comprises identifying the features' optimal regions that should be targeted by the primers included in the validation stage. The optimal regions may be made up of one or more candidate features. For example, if the data modality in use is RNAseq, exon counts could be used for the corresponding genes to identify the exons with a) the highest mean counts, and b) the largest fold changes between groups. Incorporating primer design into the discovery of the diagnostic signatures increases the likelihood of successful cross-platform validation of features. This allows primer design constraints to be considered when processing the candidate features for signature discovery.

Optionally, the method further comprises identifying optimal regions of the transcriptomic data, wherein the optimal regions are regions that are suitable to be targeted by at least one primer or probe to enable optimal discrimination when the biological condition is detected via the first test platform. The method may further comprise outputting an optimised primer design (this is described in further detail below).

At step 140, the optimised transcriptomic signature optimised for detection via the first test platform is output, based on the processing. This optimised transcriptomic signature comprises at least one feature of the plurality of candidate features. The features in the signature are ready to be translated immediately to low-throughput quantification methods.

Optionally, an optimised primer design may also be output. The method may comprise identifying at least one optimal exon, and the optimised primer design may be identified based on the at least one optimal exon. The at least one optimal exon may be identified by calculating at least one of: mean exon count for the first set of transcriptomic data, mean exon count for the first subset of subjects in the first set of subjects that do have the biological condition, and mean exon count for the second subset of subjects in the first set of subjects that do not have the biological condition.

### Requirements/Constraints

The constraints relate to requirements associated with a test platform. Requirements (which may also be referred to as ‘constraints’ herein) may arise as a result of the test platform technology itself. Additionally or alternatively, the constraints may arise as the result of the chemistry and/or the read-out method. For example, the amplification process associated with Loop-mediated Isothermal Amplification (LAMP) may result in a constraint related to the sequence target melting temperature (Tₘ).

Additionally or alternatively, the constraints may be related to the sample type, and the sample may comprise a biological fluid. A sample type may be associated with the transcriptomic data obtained from the first set of subjects, and at least one of the plurality of requirements may be based on the sample type. The sample type may be one of: saliva, whole blood, plasma, serum, lymph, synovial fluid, peritoneal fluid, pleural fluid, urine, sputum, semen, vaginal lavage, bone marrow, and cerebrospinal cord fluid and tears, among others.

Examples of constraints that are based on the instrumentation of the test platform include resolution (for example, being able to differentiate 1.2× vs 2× difference) and sensitivity (for example, the number of copies that can be seen). Further examples are reaction volume, number of fluorescent channels, the real-time vs end-point quantification, the dynamic range of quantification, and ability to perform temperature gradient. Constraints may also be based on the required preamplification step of a given test platform. They may also be based on sample preparation, or bias detection of a given target, for example based on primers.

Biases in sample preparation, sequencing, and genomic alignment and assembly can result in regions of the genome that lack coverage (that is, gaps), and also regions with much higher coverage than theoretically expected. GC-rich regions, such as CpG islands, are particularly prone to low depth of coverage partly because these regions remain annealed during amplification. Consequently, targeted PCR approaches are a good alternative when looking for very specific markers.

Examples of constraints that are based on the chemistry of the test platform include Primer and sequence target GC content (%), primer and sequence target Length (nt), sequence target Melting Temperature (Tₘ), maximum and minimum primer Tₘ, maximum and minimum primer 3′ clamp, maximum and minimum primer hairpin Tₘ, maximum and minimum primer cross-dimer Tₘ, maximum and minimum distances between primers.

For example, for sequence of interest selection for a PCR-based assay, the primer design constraints may be considered, as they relate to the assay design. This facilitates the development of accurate, specific and sensitive PCR-based assays which will optimally translate the signature finding for a prognostic molecular test.

In some examples, the requirements may be a limitation. Alternatively or additionally, the constraints may not be limiting factors. For example, the requirement might be for the melting temperature of a primer to be within a given range. A particular melting temperature for a primer targeting a particular candidate feature may not be the optimal temperature, but the feature may not necessarily be filtered out as a result of the constraint or requirement. Instead, various candidate features may be ranked according to the constraint or requirement, rather than a particular candidate feature being removed from the optimised signature entirely.

As used here, a biological ‘condition’ refers also to a biological state or a biological stage. The biological condition may be visible or detectable via transcription. For example, omics data may indicate the presence of an inflammatory state or the presence of a pathogen. The biological condition may be used as part of a diagnostic process, for example by indicating the presence of a pathogen such as viruses, bacteria, fungi or protozoa. It may additionally or alternatively be used as part of a prognostic process. For example, the biological condition as detected via transcription can be used to predict the expected development of a disease caused by a pathogen, or the development of a cancerous cell or tissue.

The transcriptomic data used to obtain the optimised transcriptomic signature may be host-response data. This data provides information on the abundance of mRNA transcripts within a biological sample. Test platforms that utilize RNA sequencing may only require a small amount of RNA, for example, single-cell RNA sequencing. In some embodiments, the transcriptomic data may be high-throughput sequencing data. In some embodiments, the data is another form of high-dimensional or high-throughput ‘omic’ data, for example measurements of proteins (proteomics), or metabolites (metabolomics).

### Feature Selection

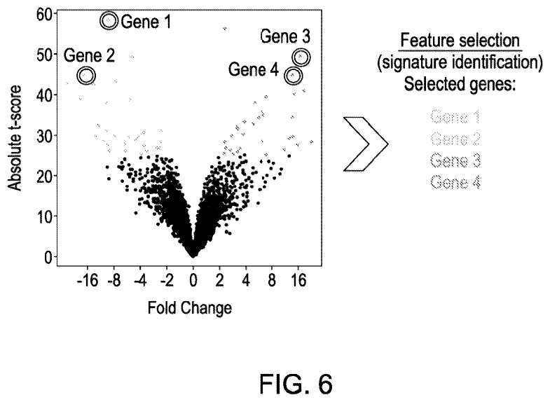

FIG. 6 depicts signature discovery based on feature selection.

Optionally, step 120 of FIG. 1 may further comprise filtering the candidate features using statistical feature selection. By filtering the genes of exons based on log-fold changes (i.e. −1 and +1), the optimal or most suitable candidate features for the RNA signature can be selected. FIG. 6 depicts the selected gene for both positive and negative fold change selected after feature extraction from RNAseq data. The x axis of FIG. 6 is the log fold change. The y axis of FIG. 6 is the absolute t-score. Each dot represents a gene of the RNAseq data, and a combination of those genes

Gene 1, gene 2, gene 3, and gene 4 are circled in this figure, with gene 1 and gene 1 being selected for negative fold change and gene 3 and gene 4 being selected for positive fold change. A plurality of statistical significance measures may be applied in this step, for example, log-fold changes and/or p-values.

### Test Platforms

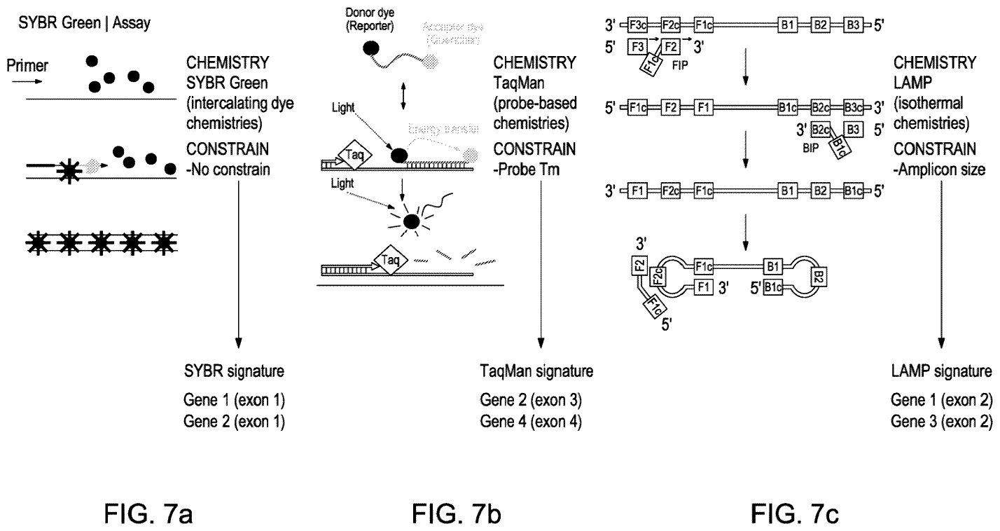

FIG. 7 depicts a pipeline for the RNA signature discovery based on different chemistries and assay design constraints.

At step 130 of FIG. 1, the plurality of candidate features may be processed based on at least one constraint associated with the test platform. The selected genes can be analysed bioinformatically by applying constraints. For example, different parameters such as GC content, Hairpin Tₘ, Amplicon length and others may be considered. The constraints might vary depending on the molecular tests we are going to perform. FIG. 7 shows different scenarios where different constraints are considered for RNA signature translation.

In FIG. 7a, the example test platform is intercalating dyes such as SYBR green. In this example, the chemistry requires the use of intercalating dye, however no constraints need to be applied to the candidate features due to the flexibility of the method.

In FIG. 7b, the example test platform is a TaqMan assay. The chemistry of this test platform requires specific constraints associated with the TaqMan probe design. For instance, the probe is required to have a melting temperature (Tₘ) 5 to 10 degrees higher than the forward and reverse primers. If the region of interest (such as an exon) presents low GC content, a suitable place for the TaqMan probe might not be available. In this example, the selection of the gene or exon for the optimised signature must accommodate a suitable primer set or sts for the signature translation in PCR test.

In FIG. 7c, the example test platform is an isothermal chemistry test platform. For example, Loop-Mediated Isothermal Amplification (LAMP) can also be used to perform molecular test and retrieve a cycle threshold (Cₜ) value for the signature translation. However, LAMP constrains are more demanding relative to PCR. There is a need for a minimum of four primers and a total amplicon length of 200 bp. To develop such a molecular test, exons with less than 200 bp are not suitable for assay design. Therefore, the processing may comprise filtering those exons out of the plurality of candidate features, in order to provide an optimised transcriptomic signature considering LAMP constrains.

FIG. 7 demonstrates that each signature will have different optimal gene selection depending on the assay design constraints (requirements). This ensures that in the development of molecular tests, the region or regions of interest (in this case, exons) are suitable for the selected chemistries.

While three testing platforms and their requirements (which may be referred to as constraints herein) are depicted in FIG. 7, the skilled person will appreciate that a plurality of test platforms are available for performing diagnostic tests, and each has various advantages and disadvantages. For example, both digital polymerase chain reaction (dPCR) or quantitative polymerase chain reaction (qPCR) are test platforms available for performing diagnostic testing. However, qPCR may be more economical when processing a large number of samples, but dPCR can be more precise, for example when low fold-changes need to be detected. There will be different requirements associated with these different test platforms. Requirements may also be referred to as constraints or limitations.

Constraints or requirements may be used to filter the plurality of candidate features of the transcriptomic data. This filtering may comprise determining that a first feature of the plurality of candidate features is unsuitable, based on the first requirement, and removing the first feature from the plurality of candidate features. Additionally or alternatively, the filtering may comprise determining that a first feature of the plurality of candidate features is essential, based on the first requirement, and including the first feature in the plurality of candidate features.

Other tests include low-throughput quantification methods, contrasted with the high-throughput data that is received to be processed (such as RNASeq data).

The present application discloses processing a plurality of candidate features, such as genes which could form part of a signature, to output an optimised signature for a particular testing platform among a plurality of test platforms. The test platform for which the signature is optimised may be a platform for targeting the RNA of a biological sample. It may be an isothermal test platform such as a Loop-mediated Isothermal Amplification (LAMP) test or a non-isothermal test platform such as polymerase chain reaction (PCR) test. Both of these test platforms utilize extracted RNA from a biological sample, and the RNA is amplified. The test platform may be selected from a plurality of test platforms.

The plurality of test platforms may include test platforms that differ in their chemistry, for example, isothermal and non-isothermal test platforms. Examples of these are PCR (isothermal) and LAMP (non-isothermal) which will have different constraints associated with the different chemistries. The test platforms may additionally or alternatively differ in the platform technologies. For example, digital polymerase chain reaction (dPCR) or quantitative polymerase chain reaction (qPCR) are both forms of PCR, however the former measures the accumulation of DNA during a PCR reaction using change in intensity of a signal, and the latter measures the presence or absence of a signal in order to calculate the number of molecules in a sample. The different methods will have different constraints associated with them. The test platform may differ in the read-out method, for example, fluorescence read-out and electrochemical read-out. Further examples of non-fluorescence readout include colorimetric and pH-based signals. Data may be generated from a variety of processes and methods, during or after the amplification event (i.e. electrophoresis and sequencing approaches).

LAMP amplification is typically carried out isothermally, at around 60 to 65° C. An example of a constraint that may be associated with the LAMP test platform is therefore the sequence target melting temperature. Some features of the transcriptomic data may have a low thermostability, in other words, a low melting temperature, and therefore be unsuitable for a signature for LAMP.

In some implementations, the test platform is an amplification method. For example, the nucleic acid amplification reaction may be a nucleic acid isothermal or non-isothermal amplification method. Examples of non-isothermal test platforms include: PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR (AS-PCR), intersequence-specific PCR (ISS-PCR), whole genome amplification (WGA), inverse PCR, or thermal asymmetric interlaced PCR (TAIL-PCR)

Isothermal amplification is a form of nucleic acid amplification which does not rely on the thermal denaturation of the target nucleic acid during the amplification reaction and hence does not require multiple rapid changes in temperature. Isothermal nucleic acid amplification methods can therefore be carried out inside or outside of a laboratory environment. Examples of isothermal test platforms include: Strand Displacement Amplification (SDA), Transcription Mediated Amplification (TMA). Nucleic Acid Sequence Based Amplification (NASBA), Recombinase Polymerase Amplification (RPA).

Rolling Circle Amplification (RCA), Ramification Amplification (RAM), Helicase-Dependent Isothermal DNA Amplification (HDA), Circular Helicase-Dependent Amplification (cHDA), Loop-Mediated Isothermal Amplification (LAMP), Single Primer Isothermal Amplification (SPIA), Signal Mediated Amplification of RNA Technology (SMART), Self-Sustained Sequence Replication (3SR), Genome Exponential Amplification Reaction (GEAR) and Isathermal Multiple Displacement Amplification (IMDA), Further examples of such amplification chemistries are described in, for example, “Isothermal nucleic acid amplification technologies for point-of-care diagnostics: a critical review” (Pascal Craw and Wamadeva Balachandrana Lab Chip, 2012, 12, 2469-2486, DOI: 10.1039/C2LC40100B).

The plurality of test platforms may include two or more of these example test platforms, selected from a list comprising both isothermal and non-isothermal amplification methods. The optimised test platform is optimised for identification of a biological condition via a test platform selected from the plurality of test platforms.

The skilled person will be familiar with many amplification chemistries, and this disclosure is not limited to any particular chemistry or reaction. Similarly, the disclosure is not limited to any particular amplification instrument. Suitable amplification instruments include any instrument capable of real-time measurements including bulk (such as qPCR platform) or single-molecule (such as dPCR platform). The method can be used with single-channel or multi-channel instruments. For example, an instrument with 5 channels (i.e. each channel reads a different colour), may be used, in which 3 targets are multiplexed per channel, totalling 15 targets in a single reaction. Similarly, the present disclosure is not limited to any particular sensing method. Sensing methods may be (i) Fluorescent based, including probe-based (e.g. Taqman. Scorpion, FRET) or dye-based (e.g. SYBR. EvaGreen, SYTO). (ii) Colorimetric based. (iii) Electrochemical based (e.g. pH or ion based sensing).

For example, the nucleic acid amplification method may comprise polymerase chain reaction (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR (AS-PCR), intersequence-specific PCR (ISS-PCR), whole genome amplification (WGA), inverse PCR, or thermal asymmetric interlaced PCR (TAIL-PCR).

### Primer Design

Advantageously, this method outputs an optimised transcriptomic signature comprising at least one candidate feature that comprises the most optimal region for cross-platform translation. Rather than only filtering based on the performance of the genes in RNAseq (such as log fold change and p-value), further requirements may be accounted for by applying at least one constraint related to a test platform. The candidate features included in the signature are therefore optimised for that test platform. These requirements may be thresholds, limitations or criteria for genes to be considered for validation that relate to the test platform. For example, they may relate to the chemistry or the instrumentation of the test platform.

The at least one constraint may be one of: Primer and sequence target GC content (%), Primer and sequence target Length (nt), sequence target Melting Temperature (Tₘ), maximum and minimum primer Tₘ, maximum and minimum primer 3′ clamp, maximum and minimum primer hairpin Tₘ, maximum and minimum primer cross-dimer Tₘ.

Advantageously, this method does not depend on commercial primers, but rather incorporates bespoke primer design, which provides more control over the targets of interest Commercial designs of primers can also be expensive. For example, whilst there are several exons associated with LEPROT gene, the commercial LEPROT primer pair detects a region at the location 1:65.900,457-65.900,598 (BIORAD LEPROT gene, human, https://commerce.bio-rad.com/en-uk/prime-pcr-assays/assay/ghsaced0037872-primepcr-sybr-green-assay-leprot-human). By contrast, an example bespoke primer design for the same gene detects a region at the location 1:65,425,301-65,425,378 reference transcript: ENSE00003644138

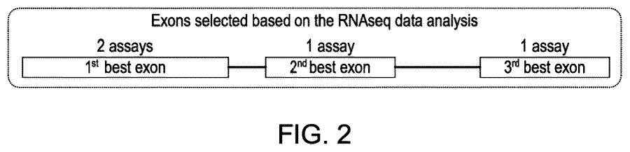

FIG. 2 depicts a workflow of exon selection based on RNAseq data analysis. It depicts a ranking of the first, second, and third best exon. One or more ssays may be designed for each of theses exons. For example, the first best exon has two assays.

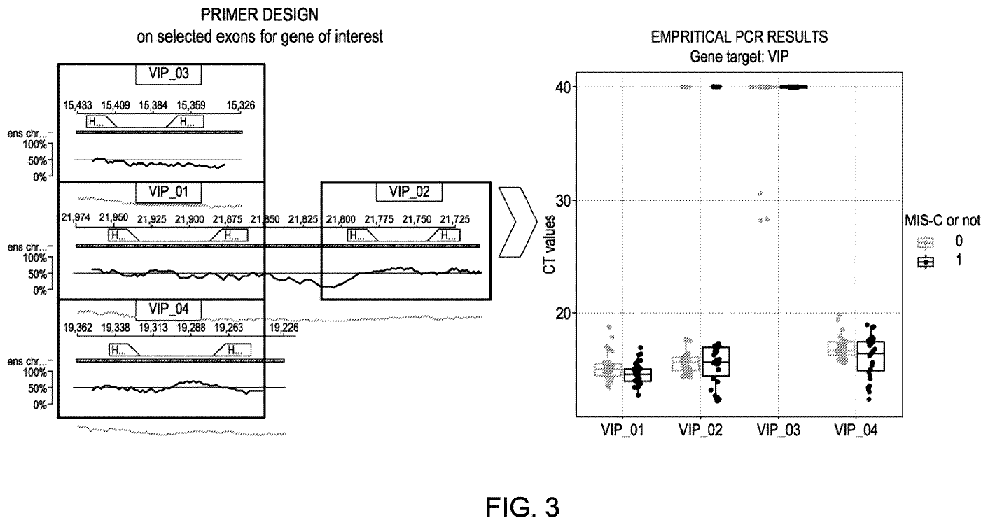

FIG. 3 depicts assay design strategy and performance in qPCR instrument. Several assays may be designed with the purpose of generating a primer pair which best detect the different expression of genes when subjects have a biological condition (in this example, Multisystem inflammatory syndrome or MIS-C) compared to subjects that do not have the biological condition. The set of subjects are children as MIS-C is a paediatric inflammatory syndrome that occurs 2-6 weeks after SARS-COV-2 infection.

There are four different assays in FIG. 3, which are named VIP_01, VIP_02, VIP_03 and VIP_04. They may be tested for the same condition with 48 clinical samples of patients with and without MIS-C diagnosis. FIG. 3 depicts the cycle threshold (Cₜ) for each of the four assays plotted in a box plot format depicting empirical PCR results. The y axis is Cₜ, and the x axis shows each of VIP_01, VIP_02, VIP_03 and VIP_04. From these plots, it can be observed that VIP_01 has good G, but the difference with MIS-C or not MIS-C samples could not be appreciated because of the Cₜ distributions. VIP_02 assay worked for majority of samples, but 7 of them didn't show a signal. VIP_03 failed for all but 3 samples. VIP_04 had good Cₜ and translated the signature as the fold-change within MIS-C or not MIS-C samples could be appreciated.

This demonstrates that not all the exons can be considered for the molecular method in PCR as primer design constraints and the sequence of the targeted nucleic acid have a significant impact in the performance of each assay.

Further, laboratory testing confirms that using standard primer design parameters do not necessary translate to the best performing assay. The present method of combining methods of processing RNAseq data and applying constraints based on the test platform (such as primer design constraints) increases the success rate of the assay. Laboratory testing also demonstrates that the primer design parameter may be adjusted according to the target to obtain improved discrimination in the presence or not of the biological event of interest.

When using conventionally designed PCR assays, the presence of the targeted region as it can be subjected to events of splicing, or their expression can be down or up regulated in certain patient with other unknown or undiagnosed conditions. Additionally, the region may be subjected of splicing not covered in the genome annotation, or genetic variation in the region may lead to failure of the primers. Using bespoke primer designs accounts for these factors.

A given gene can contain many exons, however not all exons will be optimal for cross-platform translation. Therefore, analysis of exon counts helps to improve cross-platform translation.

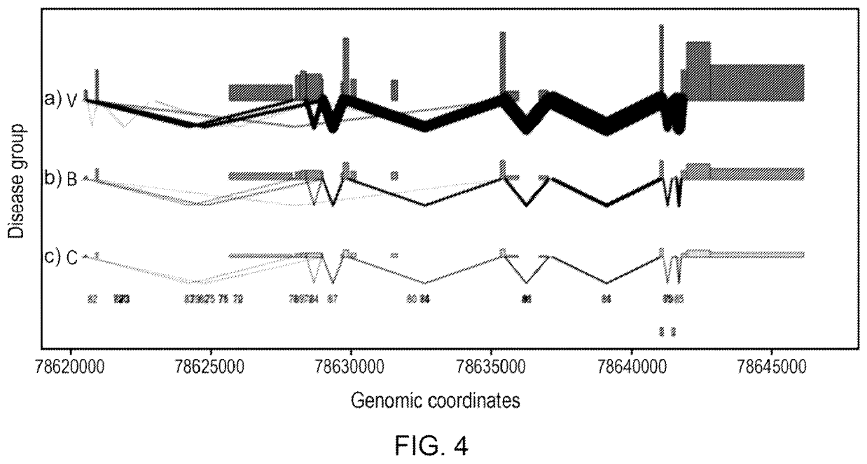

Optionally, processing the candidate features may comprise identification of the most appropriate exon. This can then be used to guide primer design. FIG. 4 depicts exon abundance for the gene biomarker of interest in the patient groups with definite viral infection shown in (a), definite bacterial infection shown in (b), and healthy controls shown in (c). The y axis shows each of these disease groups, and the x axis shows the genomic coordinates. The height of the bars indicates the abundance of the exons, and the numbers underneath the splice junctions depict the Area Under the Curve for the specific splice junction, which varies from 73% to 92%. For patients in different disease groups, exons may be present in different levels, as shown in FIG. 4, however the performance of the different exons within a gene may vary in terms of their area under the receiver operating characteristic (ROC) curve (AUC), as shown by the numbers underneath the exons represented in FIG. 4. Following the identification of a gene in a signature, the method may comprise contrasting the exons present to identify the most promising exons, considering AUC and mean counts, in addition to other parameters.

The constraints used to optimise the transcriptomic signature may be based on the optimised primer design. In some embodiments, the optimised primer design is output in additional to the optimised transcriptomic signature.

### Case Study 1

In a first example, the biological condition may be multi-system inflammatory syndrome (MIS-C). The set of subjects will comprise children as this is a paediatric inflammatory syndrome that occurs 2-6 weeks after SARS-COV-2 infection. MIS-C shares multiple clinical symptoms with other paediatric infectious and inflammatory diseases, including Kawasaki Disease (KD), and bacterial and viral infections. There is no diagnostic test for MIS-C. and diagnosis using clinical symptoms is challenging due to the highly overlapping clinical presentations of MIS-C and other infectious and inflammatory diseases.

Host gene expression has been shown to vary between different infectious and inflammatory diseases with similar clinical presentation. Small combinations of genes (diagnostic gene signatures) have been identified that can accurately distinguish between such diseases. Host gene expression profiles can be used to distinguish MIS-C from other paediatric infectious and inflammatory diseases based on gene expression. A small diagnostic gene signature for diagnosing MIS-C may be identified.

At step 110 of FIG. 1m a first set of transcriptomic data is obtained from a first set of subjects. In this first example, whole blood gene expression profiling may be performed for children with MIS-C (n=38). KD (n=136), definite bacterial infections (n=188), and definite viral infections (n=138). This set of subjects therefore includes both subjects that do have the biological condition, and those that do not have the biological condition.

At step 120, the first set of transcriptomic data is processed to identify a plurality of candidate features of the first set of transcriptomic data. In this first example, the candidate features may be candidate biomarker genes.

Genes significantly differentially expressed (SDE) between MIS-C compared to KD, MIS-C compared to DB, MIS-C compared to DV and MIS-C compared to KD+DB+DV can be identified. Feature selection using forward selection-partial least squares (FS-PLS) can be performed on the genes SDE to identify a subset of optimal combination of genes that are suitable to distinguish between MIS-C and KD+DB+DV. This subset may be small relative to the entire set of genes to be selected from.

A total of 5,696 genes are significantly differently expressed (BH-adjusted p-value <0.05) between MIS-C and the combined KD, viral and bacterial infection groups with 3,250 and 2,446 genes over- and under-expressed in MIS-C, respectively. This group of 5.696 genes may be the plurality of candidate features.

For MIS-C vs. KD, 4,786 genes were significantly differently expressed (2,681 and 2,105 genes over- and under-expressed respectively). For MIS-C vs. viral infection, 10,654 genes were significantly differently expressed (5.973 and 4,681 genes over- and under-expressed respectively). For MIS-C vs. bacterial infection 3,718 genes were SDE (1,776 and 1,942 genes over- and under-expressed respectively). TRBV11-2 was the top significantly differently expressed gene for MIS-C vs. the comparator groups combined (BH-adjusted p-value: 7.144×10-27; LFC: 1.99).

In this example, the candidate features may be selected following pre-processing and data normalisation. If the data is pre-processed, the aim is to preserve information from the transcriptomic data, and to avoid missing values or inflated variances.

In this example, the method further comprises thresholds being introduced prior to feature selection. These thresholds may be based on Benjamini-Hochberg (BH) adjusted p-value, log fold-change (LFC) and base mean counts. Only genes with BH adjusted p-values <0.001 and absolute LFC >0.5 in at least one of the following comparisons may be included: MIS-C compared to KD, MIS-C compared to DB. MIS-C compared to DV. Furthermore, genes may only be included in the plurality of candidate features if they had mean counts >50 in all disease groups and >100 mean counts in at least one disease group. This group of filtered features may instead be the plurality of candidate features

The method may further comprise applying a feature selection algorithm (such as one of FS-PLS, elastic net, LASSO, of random forest). In this example, feature selection using FS-PLS can be used to identified a four gene signature composed of HSPBAP1, VPS37C, TGFB1, and MX2. The four genes identified in the signature by the feature selection algorithm and the six most significantly differently expressed genes between MIS-C compared to KD+DB+DV can be taken forward to validation using RT-qPCR. In other words, there are a total of 10 genes which make up the plurality of candidate feature in this example.

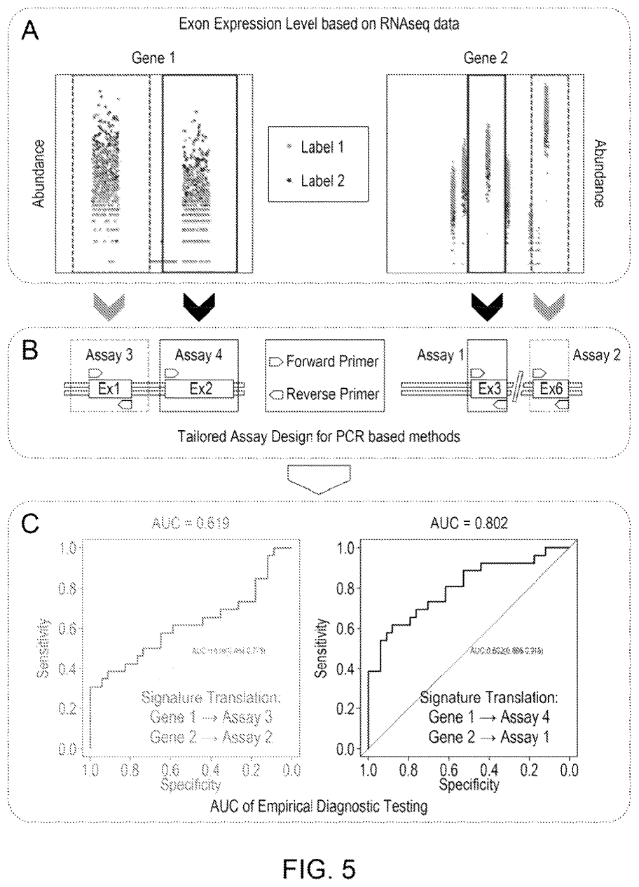

FIG. 5 depicts a schematic showing how validation platform constraints were incorporated into the signature discovery process. At box A, for each gene in the signature, all exons present in the gene were considered and the exon counts were contrasted between the group of interest vs. the comparator groups. At box B, once the exons were selected, multiple assays were designed and the performance of these assays was evaluated using metrics such as the classification accuracy or AUC. Once the optimal assays were selected, the primers for use in the validation set can be designed.

In this example, once the candidate biomarker genes are identified, the method further comprises isolating the exon counts for these genes. This is depicted in box A of FIG. 5. This step may involve identifying the features' optimal regions that should be targeted by the primers included in the validation stage. Exon count is suitable for identifying these regions because gene counts quantified by RNA-Seq includes both introns and exons. By using RT-PCR, which converts RNA into cDNA, optimal exons can be identified for each of the candidate biomarker genes.

The optimal exon may be identified for each gene target by calculating at least one of the following metrics mean exon counts overall in all disease groups; mean exon counts in MIS-C group; mean exon counts in the not MIS-C group (DB and DV): AUC for MIS-C vs. DB+DV; and LFC for MIS-C vs DB+DV. Preferably, all of these metrics are calculated in order to identify optimal exons for each candidate biomarker genes.

The exons are examined for the 10 candidate biomarker genes in this example, in order to determine where the RT-qPCR primers should target for optimal detection when RT-qPCR is the test platform. For some genes, there may be considerable differences between performance for exons. This is demonstrated in box A of FIG. 5. For example, TRBV11-2 had two exons, shown in Table 1 below which depicts exons for the candidate biomarker gene TRBV11-2. In this example, the second subset of subjects that do not have the biological condition are the DB and DV groups.

TABLE 1

| | | | Mean | Mean | | |
|---|---|---|---|---|---|---|
| | AUC (95% | Mean | exon | exon | | Exon |
| | confidence | exon | counts - | counts - | | fold |
| ID | interval) | counts | MIS-C | Not MIS-C | length | change |
| ENSE00001921870 | 59% | 2.091 | 2.45 | 1.87 | 109 | 0.389 |
| (exon 1) | (47.8%-70.3%) | | | | | |
| ENSE00002493270 | 85.9% | 48.545 | 87.39 | 24.34 | 297 | 1.844 |
| (exon 2) | (77.8%-94.1%) | | | | | |

The area under the curve (AUC) of exon 1 (ENSE00001921870) for MIS-C compared to DB+DV was 59% (95% CI: 47.8%-70.3%). This is substantially different from the performance of exon 2 (ENSE00002493270) for the same comparison, as it has an AUC of 85.9% (95% CI: 77.8%-94.1%). If the step of selecting the optimal exon was not performed, it is unlikely that TRBV11-2 would translate from RNA-Sea to RT-qPCR. Conventionally, a commercially available primer may be used which may, for example, target exon 1 rather than exon 2 and is therefore not optimal for RT-qPCR.

At step 130 of FIG. 1, the plurality of candidate features are processed based on at least one first constraint associated with a first test platform of the plurality of test platforms. In this example, the first test platforms is reverse transcription PCR (RT-PCR or RT-qPCR). This test platform may optionally be selected by user input.

In this example, for each candidate biomarker gene, 4 assays may be designed and tested on a subset of the RT-qPCR validation set. For example, each primer set may be designed aiming for a specific melting temperature (Tm), amplicon size, and GC content. These are each examples of constraints of the test platform.

For each candidate gene, the assays can be compared by analysing at least one of the following metrics: overall mean counts; mean counts for MIS-C; mean counts for not-MIS-C group; AUC for MIS-C vs. KD+DB+DV: LFC for MIS-C vs KD+DB+DV: total number of samples missing measurements.

Table 2 below shows the metrics considered for HROB, one of the genes significantly differently expressed between the subset of subjects with the biological condition (group MIS-C) compared to the subset of subjects without the biological condition (KD+DB+DV).

TABLE 2

| | Overall | MISC | KD | COVID- | DB | DV | DIAMONDS | DIAMONDS |
|---|---|---|---|---|---|---|---|---|
| | mean | mean | mean | 19 mean | mean | mean | AUC | LFC |
| HROB 1 | 6.770 | 7.222 | 6.816 | 6.338 | 6.499 | 8.076 | 55.7% | 0.378 |
| HROB 2 | 4.382 | 4.639 | 3.305 | 4.070 | 4.002 | 5.414 | 64.6% | 0.506 |
| HROB 3 | 4.097 | 4.204 | 3.092 | 3.906 | 3.917 | 4.992 | 59.3% | 0.281 |
| HROB 4 | 4.892 | 4.875 | 4.650 | 4.987 | 5.106 | 5.031 | 53.5% | −0.086 |

As shown in Table 2, the log fold change (LFC) for 3 out of the 4 assays increases in MIS-C compared to KD+DB+DV, with HROB 4 decreasing. In the discovery RNA-Seq data, HROB increased in MIS-C compared to KD+DB+DV, meaning that HROB 4 would be an unsuitable assay choice for this gene. Out of the 3 assays that increased in MIS-C, HROB 2 displayed the best performance based on area under the curve (AUC), and as a result, can be selected as the optimal assay for this gene.

This case study demonstrates that incorporating the constraints of the validation platform into the signature discovery process results in successful cross-platform translation.

At step 140, the optimised transcriptomic signature optimised for detection via the first test platform is output, based on the processing. This optimised transcriptomic signature comprises at least one feature of the plurality of candidate features. In this example, the candidate biomarker genes can be combined into a 5-gene signature (HSPBAP1, VPS370, TGFB1, MX2 and TRBV11-2), referred to as the RT-qPCR signature. This signature achieved an AUC of 93.2% (95% CI: 88.3%-97.7%) in the independent validation set when distinguishing MIS-C from KD, DB, and DV. It is therefore optimised for detection of MIS-C using RT-qPCR.

Optionally, once the optimal exons are identified for each candidate biomarker genes, at least one primer design may be output along with the optimised transcriptomic signatures. In an example, for each gene, two assays may be designed on different exons. Using clinical samples and the 4 designed assays, RT-PCR can be performed. The resulting cycle threshold (Ct) values can be used to evaluate the translation of the signature in the molecular platform. This is shown in boxes B and C of FIG. 5.

In this example, gene targets can be quantified by RT-qPCR on a set of samples from patients with MIS-C (n=36), KD (n=17), DB (n=50), DV (n=42) and COVID-19 (n=39). The performance of the candidate genes identified in the RNA-Seq discovery stage may optionally be evaluated using receiver operating characteristic (ROC) curves and area under the curve (AUC) metrics.

### A Computing Device and a Computer Readable Medium

The approaches described herein may be embodied on a computer-readable medium, which may be a non-transitory computer-readable medium. The computer-readable medium carrying computer-readable instructions arranged for execution upon a processor so as to make the processor carry out any or all of the methods described herein.

The term “computer-readable medium” as used herein refers to any medium that stores data and/or instructions for causing a processor to operate in a specific manner. Such storage medium may comprise non-volatile media and/or volatile media. Non-volatile media may include, for example, optical or magnetic disks. Volatile media may include dynamic memory. Exemplary forms of storage medium include, a floppy disk, a flexible disk, a hard disk, a solid state drive, a magnetic tape, or any other magnetic data storage medium, a CD-ROM, any other optical data storage medium, any physical medium with one or more patterns of holes, a RAM, a PROM, an EPROM, a FLASH-EPROM, NVRAM, and any other memory chip or cartridge.

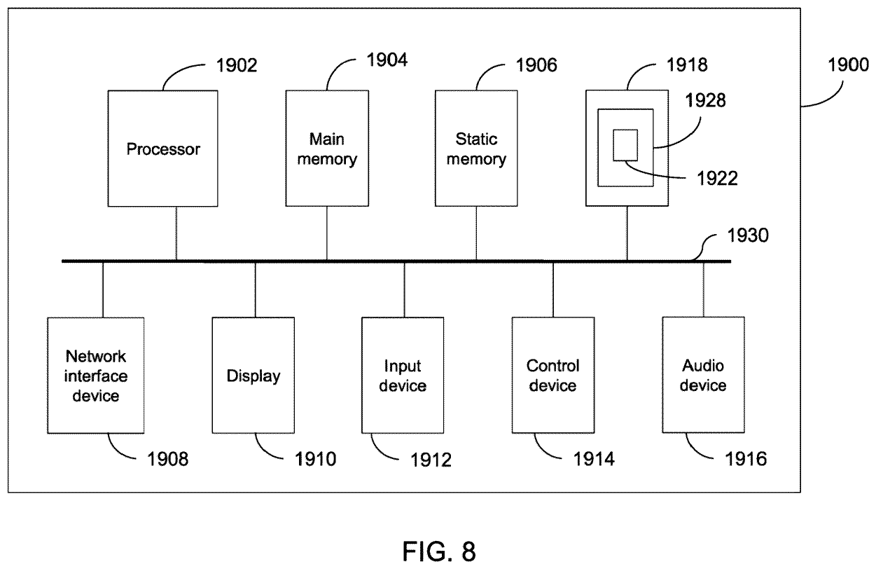

FIG. 8 illustrates a block diagram of one implementation of a computing device 800 within which a set of instructions, for causing the computing device to perform any one or more of the methodologies discussed herein, may be executed. In alternative implementations, the computing device may be connected (e.g., networked) to other machines in a Local Area Network (LAN), an intranet, an extranet, or the Internet. The computing device may operate in the capacity of a server or a client machine in a client-server network environment, or as a peer machine in a peer-to-peer (or distributed) network environment. The computing device may be a personal computer (PC), an integrated circuit, a tablet computer, a set-top box (STB), a Personal Digital Assistant (PDA), a cellular telephone, a web appliance, a server, a network router, switch or bridge, or any machine capable of executing a set of instructions (sequential or otherwise) that specify actions to be taken by that machine. Further, while only a single computing device is illustrated, the term “computing device” shall also be taken to include any collection of machines (e.g., computers) that individually or jointly execute a set (or multiple sets) of instructions to perform any one or more of the methodologies discussed herein.

The example computing device 800 includes a processing device 802, a main memory 804 (e.g., read-only memory (ROM), flash memory, dynamic random access memory (DRAM) such as synchronous DRAM (SDRAM) or Rambus DRAM (RDRAM), etc.), a static memory 806 (e.g., flash memory, static random access memory (SRAM), etc.), and a secondary memory (e.g., a data storage device 818), which communicate with each other via a bus 830.

Processing device 802 represents one or more general-purpose processors such as a microprocessor, central processing unit, or the like. More particularly, the processing device 802 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processing device 802 may also be one or more special-purpose processing devices such as an application specific integrated circuit (ASIC), a field programmable gate array (FPGA), a digital signal processor (DSP), network processor, or the like. Processing device 802 is configured to execute the processing logic (instructions 822) for performing the operations and steps discussed herein.

The computing device 800 may further include a network interface device 808. The computing device 800 also may include a video display unit 810 (e.g., a liquid crystal display (LCD) or a cathode ray tube (CRT)), an alphanumeric input device 812 (e.g., a keyboard or touchscreen), a cursor control device 814 (e.g., a mouse or touchscreen), and an audio device 816 (e.g., a speaker).

The data storage device 818 may include one or more machine-readable storage media (or more specifically one or more non-transitory computer-readable storage media) 828 on which is stored one or more sets of instructions 822 embodying any one or more of the methodologies or functions described herein. The instructions 822 may also reside, completely or at least partially, within the main memory 804 and/or within the processing device 802 during execution thereof by the computer system 800, the main memory 804 and the processing device 802 also constituting computer-readable storage media.

The various methods described above may be implemented by a computer program. The computer program may include computer code arranged to instruct a computer to perform the functions of one or more of the various methods described above. The computer program and/or the code for performing such methods may be provided to an apparatus, such as a computer, on one or more computer readable media or, more generally, a computer program product. The computer readable media may be transitory or non-transitory. The one or more computer readable media could be, for example, an electronic, magnetic, optical, electromagnetic, infrared, or semiconductor system, or a propagation medium for data transmission, for example for downloading the code over the Internet. Alternatively, the one or more computer readable media could take the form of one or more physical computer readable media such as semiconductor or solid state memory, magnetic tape, a removable computer diskette, a random access memory (RAM), a read-only memory (ROM), a rigid magnetic disc, and an optical disk, such as a CD-ROM. CD-R/W or DVD.

In an implementation, the modules, components and other features described herein can be implemented as discrete components or integrated in the functionality of hardware components such as ASICS, FPGAS, DSPs or similar devices.

A “hardware component” is a tangible (e.g., non-transitory) physical component (e.g., a set of one or more processors) capable of performing certain operations and may be configured or arranged in a certain physical manner. A hardware component may include dedicated circuitry or logic that is permanently configured to perform certain operations. A hardware component may be or include a special-purpose processor, such as a field programmable gate array (FPGA) or an ASIC. A hardware component may also include programmable logic or circuitry that is temporarily configured by software to perform certain operations.

Accordingly, the phrase “hardware component” should be understood to encompass a tangible entity that may be physically constructed, permanently configured (e.g., hardwired), or temporarily configured (e.g., programmed) to operate in a certain manner or to perform certain operations described herein.

In addition, the modules and components can be implemented as firmware or functional circuitry within hardware devices. Further, the modules and components can be implemented in any combination of hardware devices and software components, or only in software (e.g., code stored or otherwise embodied in a machine-readable medium or in a transmission medium).

Unless specifically stated otherwise, as apparent from the following discussion, it is appreciated that throughout the description, discussions utilizing terms such as “receiving”, “determining”, “comparing”, “enabling”, “maintaining,” “identifying” or the like, refer to the actions and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

It will be understood that the above description of specific embodiments is by way of example only and is not intended to limit the scope of the present disclosure. Many modifications of the described embodiments, some of which are now described, are envisaged and intended to be within the scope of the present disclosure.

### The Biological Sample and Solution

The data obtained from a first set of subjects, as described at step 110 of FIG. 1 may be any data from a suitable sample comprising one or more nucleic acids. For example, the sample may be an a clinical sample or an environmental sample. The sample may also be a sample of synthetic DNA (such as gBlocks) or a sample of a plasmid. The plasmid may include a gene or gene fragment of interest.

The subjects may be human, or non-human such as animals, microorganisms, plants, environmental samples.

An environmental sample from a surface may be from an indoor or an outdoor surface. For example, the outdoor surface be soil or compost. The indoor surface may, for example, be from a hospital, such as an operating theatre or surgical equipment, or from a dwelling, such as a food preparation area, food preparation equipment or utensils. The environmental sample may contain or be suspected of containing a pathogen. Accordingly, the nucleic acid may be a nucleic acid from the pathogen.

A clinical sample may be a sample from a patient. The nucleic acid may be a nucleic acid from the patient. The clinical sample may be a sample from a bodily fluid. The clinical sample may be from blood, serum, lymph, urine, faeces, semen, sweat, tears, amniotic fluid, wound exudate or any other bodily fluid or secretion in a state of heath of disease. The clinical sample may be a sample of cells or a cellular sample. The clinical sample may comprise cells. The clinical sample may be a tissue sample. The clinical sample may be a biopsy.

The clinical sample may be from a tumour. The clinical sample may comprise cancer cells. Accordingly, the nucleic acid may be a nucleic acid from a cancer cell.

The sample may be obtained by any suitable method. Accordingly, the method of the invention may comprise a step of obtaining the sample. The sample from a patient may contain or be suspected of containing a pathogen. Accordingly, the nucleic acid may be a nucleic acid from the pathogen. Alternatively, the nucleic acid may be a nucleic acid from the host.

The pathogen may be a eukaryote, a prokaryote or a virus. The pathogen may be found in or from an animal, a plant, a fungus, a protozoan, a chromist, a bacterium or an archaeum.

As used herein, “nucleic acid sequence” may refer to either a double stranded or to a single stranded nucleic acid molecule. The nucleic acid sequence may therefore alternatively be defined as a nucleic acid molecule. The nucleic acid molecule comprises two or more nucleotides. The nucleic acid sequence may be synthetic. The nucleic acid sequence may refer to a nucleic acid sequence that was present in the sample on collection. Alternatively, the nucleic acid sequence may be an amplified nucleic acid sequence or an intermediate in the amplification of a nucleic acid sequence.

The term “primer” as used herein refers to a nucleic acid, whether occurring naturally as in a purified restriction digest or produced synthetically, which is capable of acting as a point of initiation of synthesis when placed under conditions in which synthesis of a primer extension product, which is complementary to a nucleic acid strand, is induced, i.e. in the presence of nucleotides and an inducing agent such as a DNA polymerase and at a suitable temperature and pH The primer may be either single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the nucleic acid primer typically contains 15 to 25 or more nucleotides, although it may contain fewer or more nucleotides. According to the present invention a nucleic acid primer typically contains 13 to 30 or more nucleotides.

The nucleic acid may be isolated, extracted and/or purified from the sample prior to use in the method of the invention. The isolation, extraction and/or purification may be performed by any suitable technique. For example, the nucleic acid isolation, extraction and/or purification may be performed using a nucleic acid isolation kit, a nucleic acid extraction kit or a nucleic acid purification kit, respectively.

The method of the present disclosure may further comprise an initial step of isolating, extracting and/or purifying the nucleic acid from the sample. The method may therefore further comprise isolating the nucleic acid from the sample. The method may further comprise extracting the nucleic acid from the sample. The method may further comprise purifying the nucleic acid from the sample. Alternatively, the method may comprise direct amplification from the sample without an initial step of isolating, extracting and/or purifying the nucleic acid from the sample. Accordingly, the method may comprise lysing cells in the sample or amplifying free circulating DNA.

Following isolation, extraction and/or purification, the nucleic acid may be used immediately or may be stored under suitable conditions prior to use. Accordingly, the method of the invention may further comprise a step of storing the nucleic acid after the extracting step and before the amplifying step.

The step of obtaining the sample and/or the step of isolating, extracting and/or purifying the nucleic acid from the sample may occur in a different location to the subsequent steps of the method. Accordingly, the method may further comprise a step of transporting the sample and/or transporting the nucleic acid.

The method may further comprise diagnosing a pathogen, an infectious disease, antimicrobial resistance or a drug resistant infection if the nucleic acid molecule is present.

The infectious disease may be selected from the group consisting of Adenovirus, Coronavirus, Human Rhinovirus, Human Metapneumovirus, Parainfluenza, Respiratory Syncytial Virus, Bordetella Acute Flaccid Myelitis (AFM), Anaplasmosis, Anthrax, Babesiosis, Botulism, Brucellosis, Burkholderia mallei (Glanders), Burkholderia pseudomallei (Melioidosis), Campylobacteriosis (Campylobacter), Carbapenem-resistant Infection (CRE/CRPA), Chancroid, Chikungunya Virus Infection (Chikungunya), Chlamydia, Ciguatera, Clostridium Difficile Infection, Clostridium Perfringens (Epsilon Toxin), Coccidioidomycosis fungal infection (Valley fever), Creutzfeldt-Jacob Disease, transmissible spongiform encephalopathy (CJD), Cryptosporidiosis (Crypto), Cyclosporiasis, Dengue, 1,2,3,4 (Dengue Fever), Diphtheria, E, coli infection (E, coli), Eastern Equine Encephalitis (EEE), Ebola, Hemorrhagic Fever (Ebola), Ehrlichiosis, Encephalitis, Arboviral or parainfectious, Enterovirus Infection, Non-Polio (Non-Polio Enterovirus), Enterovirus Infection, D68 (EV-D68), Giardiasis (Giardia), Gonococcal Infection (Gonorrhea), Granuloma inguinale, Haemophilus Influenza disease, Type 8 (Hib or H-flu), Hantavirus Pulmonary Syndrome (HPS), Hemolytic Uremic Syndrome (HUS), Hepatitis A (Hep A), Hepatitis B (Hep B), Hepatitis C (Hep C), Hepatitis D (Hep D), Hepatitis E (Hep E), Herpes, Herpes Zoster, zoster VZV (Shingles), Histoplasmosis infection (Histoplasmosis), Human Immunodeficiency Virus/AIDS (HIV/AIDS), Human Papillomarivus (HPV), Influenza (Flu), Legionellosis (Legionnaires Disease), Leprosy (Hansens Disease), Leptospirosis, Listeriosis (Listeria), Lyme Disease, Lymphogranuloma venereum infection (LVG), Malaria, Measles, Meningitis, Viral (Meningitis, viral), Meningococcal Disease, Bacterial (Meningitis, bacterial), Middle East Respiratory Syndrome Coronavirus (MERS-COV), Mumps, Norovirus, Paralytic Shellfish Poisoning (Paralytic Shellfish Poisoning, Ciguatera), Pediculosis (Lice, Head and Body Lice), Pelvic Inflammatory Disease (PID), Pertussis (Whooping Cough), Plague; Bubonic, Septicemic, Pneumonic (Plague), Pneumococcal Disease (Pneumonia), Poliomyelitis (Polio), Powassan, Psittacosis, Pthinasis (Crabs; Pubic Lice Infestation), Pustular Rash diseases (Small pox, monkeypox, cowpox), Q-Fever, Rabies, Ricin Poisoning, Rickettsiosis (Rocky Mountain Spotted Fever), Rubella, Including congenital (German Measles), Salmonellosis gastroenteritis (Salmonella), Scabies Infestation (Scabies), Scombroid, Severe Acute Respiratory Syndrome (SARS), Shigellosis gastroenteritis (Shigella), Smallpox, Staphyloccal Infection, Methicillin-resistant (MRSA), Staphylococcal Food Poisoning, Enterotoxin-B Poisoning (Staph Food Poisoning), Staphylococcal Infection, Vancomycin Intermediate (VISA), Staphylococcal Infection, Vancomycin Resistant (VRSA), Streptococcal Disease, Group A (invasive) (Strep A), Streptococcal Disease, Group B (Strep-B), Streptococcal Toxic-Shock Syndrome, STSS, Toxic Shock (STSS, TSS), Syphilis, primary, secondary, early latent, late latent, congenital, Tetanus Infection, tetani (Lock Jaw), Trichonosis Infection (Trichinosis), Tuberculosis (TB), Tuberculosis (Latent) (LTBI), Tularemia (Rabbit fever), Typhoid Fever, Group D, Typhus, Vaginosis, bacterial (Yeast Infection), Varicella (Chickenpox), Vibrio cholerae (Cholera), Vibriosis (Vibrio), Viral Hemorrhagic Fever (Ebola, Lassa, Marburg), West Nile Virus, Yellow Fever, Yersenia (Yersinia), Zika Virus Infection (Zika) and COVID-19.

It is to be understood that the above description is intended to be illustrative, and not restrictive. Many other implementations will be apparent to those of skill in the art upon reading and understanding the above description. Although the present disclosure has been described with reference to specific example implementations, it will be recognized that the disclosure is not limited to the implementations described, but can be practiced with modification and alteration within the spirit and scope of the 10 appended claims. Accordingly, the specification and drawings are to be regarded in an illustrative sense rather than a restrictive sense. The scope of the disclosure should, therefore, be determined with reference to the appended claims, along with the full scope of equivalents to which such claims are entitled.

1. A computer-implemented method of obtaining an optimised transcriptomic signature for identification of a biological condition, the optimised transcriptomic signature being optimised for detection via a particular test platform of a plurality of test platforms, each test platform being associated with at least one requirement, the method comprising:
receiving transcriptomic data obtained from a first set of subjects, wherein a first subset of subjects in the first set of subjects do have the biological condition, and a second subset of subjects in the first set of subjects do not have the biological condition;
processing the transcriptomic data to identify a plurality of candidate features, each candidate feature being suitable for use in identifying the biological condition;
processing, based on at least one first requirement associated with a first test platform of the plurality of test platforms, the plurality of candidate features; and
outputting, based on the processing of the plurality of candidate features, the optimised transcriptomic signature for identification of a biological condition being optimised for detection via the first test platform, the optimised transcriptomic signature comprising at least one feature of the plurality of candidate features.

2. The method of claim 1, wherein processing the plurality of candidate features of the transcriptomic data comprises:
filtering the candidate features based on the at least one first requirement.

3. The method of claim 2, wherein the filtering further comprises:
determining that a first feature of the plurality of candidate features is unsuitable based on the first requirement; and
based on the determining, removing the first feature from the plurality of candidate features.

4. The method of claim 2, wherein the filtering further comprises:
determining that a first feature of the plurality of candidate features is essential, based on the first requirement; and
based on the determining, including the first feature in the plurality of candidate features.

5. The method of claim 1, wherein at least one of the plurality of requirements is based on instrumentation of the test platform.

6. The method of claim 5, wherein the at least one requirement relates to at least one of: resolution, depth coverage, sensitivity, reaction volume, number of fluorescent channels, real-time quantification, end-point quantification, dynamic range of quantification, ability to perform temperature gradient, required preamplification step, sample preparation, bias detection of a given target.

7. The method of claim 1, wherein at least one of the plurality of requirements is based on chemistry of the test platform.

8. The method of claim 7, wherein the at least one requirement relates to at least one of: primer and sequence target GC content, primer and sequence target length, sequence target melting temperature, maximum and minimum primer melting temperature, maximum and minimum primer 3′ clamp, maximum and minimum primer hairpin melting temperature, maximum and minimum primer cross-dimer melting temperature, maximum and minimum distances between primers.

9. The method of claim 1, wherein a sample type is associated with the transcriptomic data obtained from the first set of subjects, and at least one of the plurality of requirements is based on the sample type.

10. The method of claim 1, wherein the first test platform comprises an amplification method.

11. The method of claim 10, wherein the plurality of test platforms includes at least two of: (PCR), reverse transcription PCR (RT-PCR), quantitative PCR (qPCR), reverse transcription qPCR (RT-qPCR), nested PCR, multiplex PCR, asymmetric PCR, touchdown PCR, random primer PCR, hemi-nested PCR, polymerase cycling assembly (PCA), colony PCR, ligase chain reaction (LCR), digital PCR, methylation specific-PCR (MSP), co-amplification at lower denaturation temperature-PCR (COLD-PCR), allele-specific PCR (AS-PCR), intersequence-specific PCR (ISS-PCR), whole genome amplification (WGA), inverse PCR, or thermal asymmetric interlaced PCR (TAIL-PCR), Strand Displacement Amplification (SDA), Transcription Mediated Amplification (TMA), Nucleic Acid Sequence Based Amplification (NASBA), Recombinase Polymerase Amplification (RPA), Rolling Circle Amplification (RCA), Ramification Amplification (RAM), Helicase-Dependent Isothermal DNA Amplification (HDA), Circular Helicase-Dependent Amplification (cHDA), Loop-Mediated Isothermal Amplification (LAMP), Single Primer Isothermal Amplification (SPIA), Signal Mediated Amplification of RNA Technology (SMART), Self-Sustained Sequence Replication (3SR), Genome Exponential Amplification Reaction (GEAR) and Isothermal Multiple Displacement Amplification (IMDA).

12. The method of claim 1, wherein processing the transcriptomic data to identify a plurality of candidate features comprises filtering the plurality of candidate features based on statistical significance measures.

13. The method of claim 1, wherein processing the transcriptomic data to identify a plurality of candidate features comprises applying a feature selection algorithm.

14. The method of claim 1, wherein the transcriptomic data comprises one of: RNA-sequencing data, gene counts, exon counts, or microarray data.

15. The method of claim 1, wherein the first test platform is an amplification method, and wherein processing the plurality of candidate features of the transcriptomic data comprises:
wherein the candidate features comprise optimal regions of the transcriptomic data, wherein the optimal regions are regions that are targetable by at least one primer or probe to enable optimal discrimination when the biological condition is detected via the first test platform.

16. The method of claim 15, further comprising outputting an optimised primer design based on the at least one primer or probe.

17. The method of claim 1, wherein the optimised transcriptomic signature is optimised for at least one of efficiency, specificity, and accuracy when the biological condition is detected via the first test platform.

18. The method of claim 1, wherein the optimised transcriptomic signature is for one of the group consisting of: diagnosing a disease, prognosis of a disease, and screening for a disease.

19. (canceled)

20. A computer-readable medium comprising computer-readable instructions which, when executed by a processor, cause the processor to perform the method according to claim 1.

21. A system comprising:
a memory for storing computer-readable instructions; and
one or more processors for executing the computer readable instructions to perform the method of claim 1.
